(19) United States
(12) Patent Application Publication
(10) Pub. No.: US 2014/0147472 A1
(43) Pub. Date: May 29, 2014

(54) BIOADHESIVE COMPOSITION AND DEVICE FOR REPAIRING TISSUE DAMAGE

(71) Applicants: Nissan Elimelech, Be'erotaim (IL); Ana Dotan, Ramat-Gan (IL); Amos Ophir, Zikhron-Yaakov (IL)

(72) Inventors: Nissan Elimelech, Be'erotaim (IL); Ana Dotan, Ramat-Gan (IL); Amos Ophir, Zikhron-Yaakov (IL)

(73) Assignee: MEDIZN TECHNOLOGIES LTD., Tel Aviv (IL)

(21) Appl. No.: 13/825,609

(22) PCT Filed: Sep. 27, 2011
(86) PCT No.: PCT/IL11/00765

§ 371 Date: Jun. 7, 2013

Related U.S. Application Data
(60) Provisional application No. 61/387,152, filed on Sep. 28, 2010.
(60) Provisional application No. 61/491,366, filed on May 31, 2011.

(51) Int. Cl. A61K 31/79; A61K 31/765

(52) U.S. Cl. CPC A61K 31/79; A61K 31/765
USPC 424/400; 424/78.38; 424/78.32

(57) ABSTRACT

Provided is a bioadhesive composition and device including same, the composition including a polymeric matrix and at least one synthetic bioadhesive polymer carried by the polymeric matrix, the polymeric matrix including at least one synthetic thermoplastic polymer characterized by one or more of (a) an average molecular weight in the range of between 20,000 Da to 90,000 Da; and (b) it includes polycaprolactone (PCL); wherein exposure to heat causes the bioadhesive composition to transform into a non-solid state and to cohesively adhere to a biological tissue upon subsequent cooling thereof. Also provided herein are methods of preparing the composition or device, and use of the composition or device in therapy, e.g. for treating hernia.

### FIELD OF THE INVENTION

This invention generally relates to heat activated bioadhesive compositions and devices.

### BACKGROUND OF THE INVENTION

There are several techniques for fixation and repairing of tissue defects. The traditional technique involves the use of stitches which are tightly fixed onto the defected tissue. This technique in known to be involved with the application of tension on to the defected tissue. Other methods have evolved, including the attachment of a mesh to the tissue, onto which new tissue is grown and/or the use of biological glues.

A mesh for repairing tissue defects it typically attached to the tissue using sutures, stitches, clamps, staplers, bio-adhesives, etc. In addition, energy based methods, capable of enhancing tissue repair are described.

For example, U.S. Pat. No. 6,257,241 describes a surgical repair method of tissue by using a prosthetic device which is fixed to the tissue by application of a series of ultrasonic (US) and radio frequency (RF) energies.

U.S. Pat. No. 6,287,344 describes fixation of a prosthetic device using the combination of pressure and US energy.

In addition, the use of biomaterials in combination with radiation has been described. For example, US Patent Application Publication No. 2005/0021026 describes a method of attaching biomaterials such as elastin or elastin based biomaterials, collagen-based biomaterials, and fibrin-based biomaterials to tissue using RF radiation.

In addition, U.S. Pat. No. 5,972,007 describes a method for repairing a tissue defect using a collagen pad placed on the tissue surrounding the defected tissue together with a prosthetic which is placed over the defect and the collagen pad. Pressure and energy (RF radiation, ultrasound (acoustic/mechanical) energy, laser (coherent light) energy, ultraviolet light (electro-magnetic) energy, microwave (electro-magnetic) energy, white light (non-coherent light) energy or any combination of same) are then applied to the prosthetic at the collagen pad until the tissue and the collagen pad adhere to each other.

US Patent Application Publication No. 2003/0216729 describes methods, devices and compositions to conductively or to inductively fix substrates, including tissues, using electromagnetic energy. Also described in this publication is a method of controlling the fixing process via feedback monitoring of a property of the composition and/or of the electromagnetic energy used.

Further, PCT Application Publication No. WO2007/126906 describes the use of an implant which may be electrically activated to adhere to tissue by activating a thermally crosslinkable material (e.g. albumin) that is in contact with an electrically conductive structure. For example, an implant may be coated with a thermally crosslinkable material.

Yet further, Benson R S [Benson R S, Nuclear Instruments and Methods in Physics Research B 191:752-757 (2002)] describes the use of radiation in biomaterials science. Benson R S refers to Kao et al. [Kao F J et al. Appl. Biomaterl. 38:191 (1997)] which describes the preparation of a series of UV curable bio-adhesive based on N-vinylpyrrolidine, which adhere well to tissues and were all found suitable for wound closure.

Finally, U.S. Pat. No. 5,895,412 describes a method and device for sealing a wound, using as a sealant material comprising preferably a combination of at least one biological polymer such as collagen and a synthetic organic polymer. The sealant material is heated before being applied to the wound.

### SUMMARY OF THE INVENTION

The present disclosure provides, in accordance with a first of its aspects, a bioadhesive composition comprising a polymeric matrix and at least one synthetic bioadhesive polymer carried by the polymeric matrix, the polymeric matrix comprising at least one synthetic thermoplastic polymer characterized by one or more of the following:

(a) an average molecular weight in the range of between 20,000 Da to 90,000 Da;

(b) it comprises polycaprolactone (PCL);

wherein exposure to heat causes the bioadhesive composition to transform into a non-solid state and to cohesively adhere to a biological tissue upon subsequent cooling thereof.

In accordance with a second aspect, the present disclosure provides a method for producing a bioadhesive composition comprising mixing a mixture comprising at least one synthetic thermoplastic polymer suitable for forming a polymeric matrix and at least one synthetic bioadhesive polymer under conditions at which the at least one synthetic thermoplastic polymer transforms into a non-solid state, the at least one synthetic thermoplastic polymer being characterized by one or more of the following:

(a) an average molecular weight in the range of between 20,000 Da to 90,000 Da;

(b) it comprises polycaprolactone (PCL);

the bioadhesive composition being such that when in a non-solid state it is capable of cohesively adhering to a biological tissue.

In accordance with a third aspect, the present disclosure provides a bioadhesive device comprising a support structure and a bioadhesive composition disclosed herein, wherein at least a portion of the support structure is coated or impregnated with the bioadhesive composition.

Yet, in accordance with a fourth aspect, the present disclosure provides a therapeutic method comprising placing a bioadhesive composition or a bioadhesive device as disclosed herein onto or into a biological tissue region, the tissue region comprising tissue damage or a region in predisposition of developing tissue damage;

wherein placing of the bioadhesive composition or the bioadhesive device comprises allowing contact of the bioadhesive composition with the biological tissue region while the bioadhesive composition is heated to a temperature that causes the composition to be in a non-solid state and once in contact with the biological tissue region, allowing the bioadhesive composition to cool to the temperature of the biological tissue region thereby providing cohesive adherence of the bioadhesive composition or the bioadhesive device to the biological tissue region.

Finally and in accordance with a fifth aspect, the present disclosure provides a kit comprising a bioadhesive composition or a bioadhesive device as disclosed herein, and instructions for use of the bioadhesive composition or the bioadhesive device in combination with heat energy for adherence to a biological tissue region.

### DETAILED DESCRIPTION OF EMBODIMENTS

The present disclosure relates to thermal-responsive materials and is based on the development of novel synthetic bioadhesive compositions capable and suitable for fixating biological tissue.

Specifically, and in accordance with a first aspect, the invention provides a bioadhesive composition comprising a polymeric matrix and at least one synthetic bioadhesive polymer carried by the polymeric matrix, the polymeric matrix comprising at least one synthetic thermoplastic polymer characterized by one or more of the following:
an average molecular weight in the range of between 20,000 Da to 90,000 Da;
it comprises polycaprolactone (PCL);

wherein exposure to heat causes the bioadhesive composition to transform into a non-solid state and to cohesively adhere to a biological tissue upon subsequent cooling thereof.

In the context of the present disclosure, the term “bioadhesive composition” is used herein to denote an mixture of one or more thermoplastic polymer and one or more synthetic bioadhesive polymer, which under physiological environment is capable of stably adhering to biological tissue. Stable adherence should be understood as fixation to the tissue to an extent sufficient to receive the medical purpose of fixation. The fixation should be for a sufficient time and/or to a sufficient cohesive strength that allow the composition to remain connected and not tear apart from the tissue. It is well appreciated that the higher the cohesive forces are, the stronger is the adherence. Thus, in the context of the present invention, the bioadhesive composition is at least partially cohesive, i.e. it may apply cohesive forces to the entire tissue surface in contact with the composition or it may contain areas of cohesive adherence to the surface. The level of adherence may be determined by well acceptable tests, such as peeling tests.

In general, and without being bound by theory, the chains of thermoplastic polymers are connected to the tissue via Van-der-Waals forces, dipole-diople interactions and hydrogen bonding, stacking aromatic interactions or combinations thereof.

In the context of the present disclosure, solidification includes transition into solid as well as semi- (quasi-) solid form. These processes of melting and solidifying are reversible and may be repeated.

In some embodiments, the adhesion to biological tissue is at until the composition dissolves or disintegrates. In accordance with some other embodiments, the adhesion to biological tissues may be for a time period sufficient to allow tissue regeneration and tissue growth at the area of damaged tissue.

When referring to solid state and non-solid state, it is to be understood as referring to the flowability of the composition or components thereof at room temperature (˜25° C.). When in non-solid state, the composition flows to an extent sufficient for application (e.g. spreading, pouring, introducing, or any other manner of placing) of the composition into or onto the biological tissue. Flowability may be determined by any conventional liquid or fluid flow tests such as melt flow index (MFI), capillary rheometry. At times, flowability of the composition or component thereof may be characterized by its complex viscosity, as further discussed below. At times, and without being limited thereto, a solid state of a composition would be one having a complex viscosity above 600.

In connection with the above, it is noted that the bioadhesive composition is not in the form of polymeric spheres, e.g. microspheres.

One component of the bioadhesive composition is the polymeric matrix. The polymeric matrix comprises at least a thermoplastic polymer. The thermoplastic polymer may include a single type of polymer (e.g. in terms of chemical formula of the repeating unit and average length of the polymer), a combination of polymers or a combination of one or more polymers with other additives.

Specifically, in the context of the present disclosure, the “thermoplastic polymer” also known by the term “thermoplast”, is a biocompatible polymer (e.g. degrade in vivo into non-toxic units that are eliminated from the body) that is converted to a non-solid state when heated. In other words, when heated, it softens and fluidizes (melts) albeit, once cooled, the thermoplastic polymer solidifies. The thermoplastic polymer may be re-melted and re-molded more than once (as opposed to thermosetting).

Once the bioadhesive composition reaches a temperature at which the thermoplastic polymer softens or fluidizes, the entire composition turns soft, allowing its application onto or into the tissue. This temperature may be characterized by the polymers' melt flow index (MFI). As appreciated, as the polymer's molecular weight typically increases the MFI decreases as a result of a lower melt flow (higher viscosity).

In some embodiments, it is preferable that the melting point of the thermoplastic polymer be in the range of 50° C. and 120° C., at times above 60° C., even above 70° C., and even above 85° C. In some embodiments, the phase transition temperature is not more than 100° C. Since the thermoplastic polymer forms the majority of the bioadhesive composition, these temperature ranges also apply to the point at which the composition turns “sticky” and is capable of cohesively adhering to a biological tissue. Thus, in accordance with an embodiment of the invention, the entire composition transforms into a non-solid state at a temperature between 50° C. and 100° C.

In this connection it is noted that for facilitating release of the bioadhesive polymer from the matrix it is essential that it transforms into its non-solid state (e.g. fluidizes). To this end, the thermoplastic matrix is constructed such to have a complex viscosity of between 50 to 600, even between 100 to 600 as measured with composition samples of 25 mm diameter, 2 mm thickness and test temperature of 85° C., at a frequency sweep step of 1 to 10 Hz.

In some embodiments, the thermoplastic polymer is thermosensitive to dielectric heating. In some other embodiments, the thermoplastic polymer is sensitive to high frequency alternating current. As appreciated dielectric heating, also known as electronic heating, RF heating, high-frequency heating and diathermy, is the process in which electromagnetic radiation such as radio wave or microwave heats for example a dielectric material. This heating is caused by dipole rotation.

The thermoplastic polymer is a synthetic or semi synthetic polymer and it is preferably water insoluble and/or is insoluble in bodily fluid.

The term “synthetic or semi-synthetic” polymer is used herein to denote that the polymer is not a naturally occurring polymer. The material may be a completely synthesized polymer (fully synthetic) or it may be semi-synthetic in the sense that it is a result of modification of a naturally occurring material. The modification may be a chemical modification, e.g. the insertion or deletion of one or more chemical groups, the modification may be the product of a genetically engineered variation of the naturally occurring material or any other modification known in the field of material engineering. Polymer modifications are further discussed below.

The thermoplastic polymer may be a homopolymer, a copolymer or polymer blends/mixture. Without being bound thereto, the thermoplastic polymer may be any one of a polyester, such as polycaprolactone (PCL) and polylactide (PLA), members of the polyhydroxyalkanoate family (PHA); a polyether, such as polyethylene oxide (PEO); polyglycolic acid, such as polybutylene succinate (PBS), polybutylene succinate adipate (PBSA), derivatives of natural biopolymers such as modified starch, and cellulose; thermoplastic polyurethane. The derivatives may be, for example, acetylated, hydroxypropylated, polyester-grafted derivatives, such as, thermo-plastified starch (TPS) or carboxymethylated cellulose.

In a preferred embodiment, the thermoplastic polymer is a homo or copolymer, comprising the monomer ∈-Caprolactone (also known as 2-Oxepanone). A homopolymer formed from ∈-Caprolactone monomer, is polycaprolactone (PCL).

In some embodiments, the thermoplastic polymer is a PCL copolymer selected from the group consisting of a copolymer or blend of (abbreviations provided hereinabove) PLA and PCL, a copolymer or blend of PEO and PCL, a copolymer or blend of CH and PCL, and a copolymer of TPS and PCL, or a copolymer or blend of any combination of PHA, PBS and PBSA.

At times, the thermoplastic polymer is a polymer blend comprising a combination of at least the following two polymers: PCL and PLA; PCL and PEO, PCL and PBS, PCL and PBSA, PCL and PHA and PCL and TPS.

The at least one thermoplastic polymer may be defined by a medium molecular weight (MMW) being within the range of 15,000 Da to 100,000 Da, at times between 20,000 Da to 90,000 Da, preferably between 40,000 Da to 80,000 Da or even between 40,000 Da to 50,000 Da. In some specific embodiments the at least one thermoplastic polymer may be defined by an average molecular weight of 43,000 Da-48,000 Da.

In some embodiment, the thermoplastic polymer is a combination of low molecular weight (LMW), e.g. average MW below 15,000 Da and high molecular weight (HMW) e.g. average MW above 100,000 Da, thermoplastic polymers In some other embodiments, the composition contains a single thermoplastic polymer of a medium average molecular weight.

As appreciated there are several ways of defining average molecular weight of a polymer for example. While the forgoing referred to molecular weight per se (Mw), the polymers may also be characterized by the number-average molecular weight (Mₙ).

Thus, when referring to “high molecular weight polymer” it is to be understood to encompass a thermoplastic polymer having a number-average molecular weight above 80,000 Da, at times in the range of 80,000 Da to 150,000 Da, preferably in the range of 90,000 Da to 120,000 Da or a weight-average molecular weight higher than 90,000 Da.

When referring to “low molecular weight polymer” it is to be understood to encompass a thermoplastic polymer having a weight-average molecular weight in the range 1,000 Da to 15,000 Da, at times in the range of 5,000 Da to 15,000 Da or even in the range of and a number-average molecular weight in the range of 10,000 Da.

When referring to “medium molecular weight polymer” it is to be understood to encompass a thermoplastic polymer having a weight-average molecular weight as defined above and a number-average molecular weight in the range of 40,000 Da to 50,000 Da. In some embodiments, the thermoplastic polymer comprises at least one PCL. The PCL may be a polymer of a single type (e.g. all PCL having the same average M_{w}), or a combination of PCL's. It is preferable that the PCL in the matrix has an average medium M_{w}.

At times, the LMWPCT is in combination with a HMWPCL. The combination may comprise for example 25% LMWPCL and 75% HMWPCL even 50% LMWPCL and 50% HMWPCL or preferably even 75% LMWPCL and 25% HMWPCL. It is in accordance with one embodiment that the combination of LMWPCL and HMWPCL has an average M_{w} in the MMW range.

The thermoplastic polymer may be used as the sole thermoplastic polymer constituting the polymeric matrix, or in combination with other thermoplastic polymers. Thus, in accordance with one embodiment, the matrix consists of a single thermoplastic polymer.

The polymeric matrix constitutes the majority of the bioadhesive composition and is used to carry (hold) the bioadhesive polymer whereby, under suitable conditions. e.g. temperature at which the thermoplastic polymers turns into a non-solid state, the matrix “releases” the bioadhesive polymer embedded therein or carried thereby. In this context, when referring to majority, it is to be interpreted as meaning that at least 60% (w/w of total composition) of the bioadhesive composition is made of the polymeric matrix, at times, between 60% and 80% of the total weight entire composition. The release of the bioadhesive polymer will be further discussed below.

In some embodiments, the at least one thermoplastic polymer is water insoluble or insoluble in bodily fluid. This means that the composition is essentially free of water.

The polymeric matrix may include also some additives, facilitating in the formation of the matrix. These may include, without being limited thereto plasticizers, surfactants, coupling agents, adhesion promoters, nucleating agents or fillers. The plasticizers may be used to reduce the brittleness and enhance flexibility of the bioadhesive composition. In some embodiments, the plasticizers may be at least one of tocopherol, tocopheryl-1polyethylene glycol succinate (TPGS), citrate plasticiser esters such as triethyl citrate, acetyl triethyl citrate, tributyl citrate, tributyl acetyl citrate (TAC), vitamin E (VI-E) or tri-(2-ethylhexyl)-citrate.

Fillers may be selected for example from calcium carbonate, glass fibers, talc, TiO₂ magnesiumhydroxide (M_{g}(OH)₂).

Adhesion promoters an nucleating agents may be selected, for example, from metal oxide particles, polycarbophil, carbomers and/or dextran aldehyde.

Thus, it is possible to tailor the overall matrix properties to improve mechanical and physical properties thereof such as adhesion resistance, flexibility, suitable rheological properties in the liquid (melt) state and thermal properties (low melting point). When including one or more additives, the latter will constitute not more than 10% of the components forming the matrix. Thus, when the matrix forms 60% of the bioadhesive composition, the additives will constitute no more than 6% of the total composition.

In some embodiments, the biocompatible additives mixed with the thermoplastic polymer and the bioadhesive polymer comprises at least one plasticizer and one adhesion promoter.

In a particular embodiment, the biocompatible additives are selected from at least one of alpha-tocopherol (vitamin E), polycarbophil and tributyl acetyl citrate.

The thermoplastic polymer is in a mixture with at least one synthetic bioadhesive polymer. The bioadhesive polymer constitutes about 20% to 40% (w/w) of the total bioadhesive composition.

The term “bioadhesive polymer” in the context of the present invention includes any synthetic or semi synthetic biocompatible (e.g. degrade in vivo into non-toxic units that are eliminated from the body) polymer, including from dimmers to oligomers. The art includes various biocompatible adhesives polymers that may be used in the context of the invention, such as those belonging to biocompatible acid anhydride polymers, polyurethanes, polyacrylates, polysaccharides, polyvinyl alcohol (PVOH), polyvinyl acetate, polyvinylpyrrolidone (PVP), epoxy, polyesters, polyethylene glycol (PEG) and bioadhesive amino resins.

In one embodiment, the bioadhesive polymer is polyvinylpyrrolidone (PVP), at times also referred to as polyvidone or povidone. PVP is characterized by its ability to bind to polar molecules, owing to its polarity. In the context of the present invention, the PVP has a molecular weight above 10,000 Da. At times, the PVP has a molecular weight of between 10,000 Da and 100,000 Da, preferably between 30,000 Da and 70,000 Da, more preferably between 40,000 Da and 60,000 Da.

In one embodiment of particular interest, the bioadhesive composition comprises PCL, preferably MMWPCL (optionally including additives) and PVP.

Alternatively or in addition, the bioadhesive polymer may be an acid anhydride polymer (or oligomer). Herein, when referring to acid anhydride it is to be understood as encompassing acid anhydride oligomer as well as acid anhydride polymer. The acid anhydride comprises a diacide or a polydiacide linked by anhydride bonds.

In the context of this embodiment, the acid anhydride has a molecular weight of between 100 Da and 5,000 Da and may be formed in a reflux reaction of the diacid with excess acetic anhydride. The excess acetic anhydride is evaporated under vacuum, and the resulting oligomer (or polymer), which is a mixture of species which include between about one to twenty diacid units linked by anhydride bonds, is purified by recrystallizing, for example from toluene or other organic solvents. The acid anhydride is collected by filtration, and washed, for example, in ethers the reaction produces anhydride oligomers of mono and poly acids with terminal carboxylic acid groups linked to each other by anhydride linkages. The presence of an anhydride bond in the bioadhesive composition may be detected by the presence of anhydride bonds using Fourier transform infrared spectroscopy by the characteristic double peak at 1750 cm⁻¹ and 1820 cm⁻¹, with a corresponding disappearance of the carboxylic acid peak normally at 1700 cm⁻¹.

The acid anhydride oligomer or polymer is hydrolytically labile. This can be analyzed by gel permeation chromatography. The anhydride bonds can be detected by Fourier transform infrared spectroscopy by the characteristic double peak.

In some embodiments, the acid anhydride is the oligomer, having a molecular weight of between 100 Da and 5,000 Da.

In some embodiments, the acid anhydride oligomer comprises no more than 20 diacid monomers linking the anhydride monomers in the anhydride oligomer.

In some other embodiments, the acid anhydride is in a polymeric form linked by anhydride bonds and having carboxy end groups linked to a monoacid by anhydride bonds. The diacid may be a dicarboxylic acid selected from the group consisting of saturated (aliphatic) dicarboxylic acids, aromatic dicarboxylic acids or unsaturated dicarboxylic acids.

The diacid may be selected from the group consisting of fumaric acid (FA), maleic acid, sebacic acid, oxalic acid, malonic acid, succinic acid, glutaric acid, adipic acid (AA), pimelic acid, suberic acid, azelaic acid, ortho-phthalic acid, isophthalic acid, terephthlic acid, 1,3 bis(p-carboxyphenoxy)propane (CPP), 1,6 bis(p-carboxyphenoxy)hexane (CPH), 1,4-phenylene dipropionic acid (PDP), or dodecanedioic acid (DD).

In a particular embodiment, the diacid is at least FA and thus the bioadhesive polymer is fumaric anhydride oligomer (FAO).

The present disclosure also concerns a method of producing the bioadhesive composition. The method comprises mixing a mixture comprising at least one synthetic thermoplastic polymer suitable for forming a polymeric matrix and at least one synthetic bioadhesive polymer under conditions at which the at least one synthetic thermoplastic polymer transforms into a non-solid state, the at least one synthetic thermoplastic polymer being characterized by one or more of the following:
an average molecular weight in the range of between 20,000 Da to 90,000 Da;
it comprises polycaprolactone;

to form the bioadhesive composition, the bioadhesive composition being as defined herein above.

In accordance with the preparatory method, the conditions may be any that allow the formation of an essentially homogenous (typically flowing) mixture of the components (typically as determined by visual inspection of the components being mixed). For example, the conditions may comprise mixing of a mixture at a temperature above the melting point of the thermoplastic polymer(s), e.g. at a temperature of between 50° C. and 100° C. In yet some other embodiments the conditions comprise dissolving the at least one thermoplastic polymer in an organic solvent, preferably an organic polar aprotic solvent. Examples for polar aprotic solvents include dimethyl sulfoxide, dimethylformamide, dioxane and hexamethylphosphorotriamide, acetone, tetrahydrofuran, chloroform, and ethyl acetate. The solvents may be used in combination, as well as in stages.

An example of an organic that was found suitable for mixing PCL and PVP is dichloromethane.

Once an essentially homogenous mixture is formed, the composition is allowed to cool to room temperature, whereby a solid composite is formed. Cooling may be on a support structure, in a mold or in an applicator.

In one embodiment, cooling in the mold provides laminates of the bioadhesive composition. In some embodiments, the mold is in a form allowing the formation of an essentially perforated solid structure, e.g. a net-like laminate. A perforated configuration is typically required when heating by diathermia. The laminate may be of any size or dimension, according to the particular need.

In some other embodiments, the composition is cooled in a mold providing solid cylindrical sticks of the bioadhesive composition. Such sticks may be used commercially available bioadhesive applicators. A typical applicator includes housing, a heat sink and tip assembly and a cartridge assembly, the latter carrying the bioadhesive composition, and a plunger assembly for advancing the adhesive composition into the heat sink. The heat sink and tip assembly is attached to the front of the housing for melting and dispensing the bioadhesive composition onto the surface area of the biological tissue in need thereof.

As noted above, the bioadhesive composition may be cooled on a support structure or placed on a supported structure at a later stage (e.g. prior to use). The association between the composition and the support structure may require re-heating of the composition. The support structure may be any biocompatible material and at times biodegradable material, for carrying the bioadhesive composition. Carrying may include any form of association between the support structure and the composition, including, impregnating or soaking the support structure with the composition (when the latter is in fluid form), coating (including extrusion coating) a surface of the support structure, spraying, lamination, layering over. The support structure may be in any form, including, for example, and without being limited thereto, biocompatible synthetic fibers such as gauze, dressing, bandage, graft and mesh (such as those used in hernia).

The support structure may be, in accordance with some particular embodiments, a polypropylene mesh. The polypropylene may be for example a monofilament polypropylene.

In some embodiments, the polypropylene may be used in combination with additional materials such as for example: poliglecarpone-25, oxidized regenerated cellulose, polytetrafluoroethylene (PTFE), expanded PTFE (ePTFE), silicon or polyvinylidene Fluoride (PVDF).

The mesh may be made from other biocompatible materials such as prolene, surgipro, trelex, atrium, merselene polyglactin (vicryl) polyglycolic acid (dexon), polyester, polyethylene glycol, glycerol, PTFE or ePTFE or combinations thereof.

The combination of the support structure and the bioadhesive composition forms a bioadhesive device. Upon exposure of the device to heat (either directly and/or indirectly (by heating the tissue)), the associated bioadhesive composition provides medically stable (cohesive) fixation of the mesh onto the damaged tissue.

The bioadhesive composition/device is for fixation onto bodily tissue, so as to repair tissue damage. The biological (bodily) tissue may be selected from, without being limited thereto, mucosal tissue, epithelial tissue, connective tissue, muscle tissue, blood vessel tissue (e.g. endothelium tissue) and nervous tissue. In one preferred embodiment, the biological tissue is a mucous or epithelial tissue. The bodily tissue is typically a tissue region comprising tissue damage. The tissue damage may be selected from tissue protrusion, tissue weakening and tissue rupture.

Thus, in the context of the invention, the “tissue region” may be any region of a biological tissue as defined hereinabove, possibly containing a damaged area or an area in predisposition of being ruptured or otherwise damaged or defected. Predisposition may be based on early prognosis, genetic tendency, medical history etc.

A “tissue damage” or “tissue defect” may include any type of damage to tissue that may include tissue protrusion, tissue deformation, tissue weakening, tissue hole or rupture. The tissue defect may occur in any anatomical portion of the body. In one embodiment, the tissue damage is a hernia, including various types of hernia, such as, without being limited thereto, Inguinal hernia, femoral hernia, umbilical hernia, incisional hernia, diaphragmatic hernia (hiatus hernia), congenital diaphragmatic hernia, Morgagni's hernia, Cooper's hernia, epigastric hernia: Littre's hernia: Lumbar hernia, Petit's hernia, Grynfeltt's hernia, Maydl hernia, Obturator hernia, Pantaloon hernia, Paraesophageal hernia, Paraumbilical hernia, Perineal hernia, Properitoneal hernia, Richter's hernia, Sliding hernia, Sciatic hernia, Spigelian hernia, sports hernia, Velpeau hernia or Amyand's hernia.

In some other embodiments, the tissue may be a blood vessel tissue or external (e.g. skin) wound. According to this embodiment, the “tissue damage” or “tissue defect” may be related to bleeding of blood vessels for example small blood vessels.

Upon placing of the composition or device onto the tissue region, the composition or device is exposed to heat energy. In this connection “heating” or “exposure to heat energy” is used to denote the application of thermal heat or high frequency electromagnetic currents.

As appreciated, heat may be generated from different types of energy for example such as electrical energy, mechanical energy, chemical energy, nuclear energy, sound energy and thermal energy itself which are converted to heat energy.

In one embodiment, the heat is obtained using alternating current (AC) for example high frequency AC or direct current (DC). Heat may also be generated using ultrasonic or radiofrequency devices that are used to heat a thin wire (filament). Examples of commercially available devices include, without being limited thereto, Harmonics™, LigaSure™ surgical devices as well as the electrosugery device known as “bovie” or Starion devices.

The heat is applied onto the bioadhesive composition or onto the bioadhesive device (when in situ) or onto the tissue region being in contact with the composition or device, all of which eventually causing heating of the bioadhesive composition. The heat energy is applied such as to be insufficiently intense to destroy tissue or to impair tissue vitality, but sufficient to cause the at least partial fluidizing of at least the thermoplastic polymer in the bioadhesive composition which results in the release of the bioadhesive from the composition and adherence of the composition or device to the tissue region. As an example, the exposure to heat energy is by heat conduction, e.g. from a metal probe heated by electric current.

Heat energy may be applied on at least one location (portion) of the bioadhesive composition or of the bioadhesive device (direct heating of the bioadhesive composition/device) or of the tissue region or of the surroundings (indirect heating of the composition/device).

According to some embodiments, placing the bioadhesive composition or device may comprise holding the bioadhesive composition directly in contact with the tissue region while applying heat.

In one embodiment, the heat is applied on the tissue region which in turn heats the bioadhesive composition. Alternatively, heating may include direct heating of the bioadhesive composition. Heating may be continuous or pulsed heating.

In one embodiment, the application of heat is by using dielectric energy applied on the tissue region. According to some other embodiments, application of heat energy is by the use of electrocautery (elecrosurgery) devices, also known by the name diathermy device suitable known to be used in surgical rooms.

Heating may be achieved using a hot plate, a harmonic heating device and bipolar surgical devices, an applicator in the form of a hot glue gun as well as any other device creating direct or residual heat.

At any rate, upon heating, at least a portion of the bioadhesive composition melts and at least partially migrates towards the biological tissue, at which point, it is then cooled again, to form the cohesive fixation effect.

Specifically for hernia, the therapeutic method provides the surgeon with an improved attachment of a support structure such as a mesh or a graft on a damaged tissue region by an application of heat energy without applying mechanical force. Currently, methods used in hernia surgery use either mechanical fixation of a mesh or alternatively use biological adhesives which are less convenient for use. In this connection, it is noted that biological adhesives are typically fluid at room temperatures, which render their use complicated.

Finally, there is provided a kit comprising a bioadhesive composition and instructions for use of same for the preparation of a bioadhesive device.

Alternatively, there is provided a kit comprising a bioadhesive device and instructions for use of same in treatment of tissue damage.

The kit may also include a heating unit, such as, diathermy device, a hot glue gun type applicator or a hot plate.

As used herein, the forms “a”, “an” and “the” include singular as well as plural references unless the context clearly dictates otherwise. For example, the term “a thermoplastic polymer” includes one or more polymers.

Further, as used herein, the term “comprising” is intended to mean that the composition includes the recited components, i.e. a thermoplastic polymer and an acid anhydride oligomer, but not excluding other elements, such as adhesion promoters/nucleating agents and other additives. The term “consisting essentially of” is used to define compositions which include the recited components but exclude other elements. “Consisting of” shall thus mean excluding more than trace amounts of other elements.

Further, all numerical values, e.g. when referring the amounts or ranges of the components constituting the composition are approximations which are varied (+) or (−) by up to 20%, at times by up to 10% of from the stated values. It is to be understood, even if not always explicitly stated that all numerical designations are preceded by the term “about”.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### DETAILED DESCRIPTION ON SOME NON-LIMITING EXAMPLES

### Materials and Devices

Fumaric acid (Sigma Aldrich, CAS no. 111-17-8).
Acetic anhydride (Sigma Aldrich, CAS no. 108-24-7)
Low-Molecular-Weight polycaprolactone (LMWPCL) (Sigma Aldrich, Mw˜14,000 Da, Mn˜10,000 Da)
High Molecular-Weight polycaprolactone (HMWPCL) (Sigma Aldrich, Mn˜70,000 Da˜90,000 Da)
Medium Molecular-Weight polycaprolactone (MMWPCL) (Sigma Aldrich, Mn˜45,000 Da, M_{w} 48,000 Da −90,000 Da)
Low-Molecular-Weight Polyvinyl pyrrolidone (LMWPVP) (Sigma Aldrich, average M_{w}˜10,000 Da)
Medium Molecular-Weight Polyvinyl pyrrolidone (MMWPVP) (Sigma Aldrich average M_{w}˜40,000 Da)
High Molecular-Weight Polyvinyl pyrrolidone (HMWPVP) (Sigma Aldrich average M_{w}˜60,000 Da)
Polyvinyl alcohol (PVOH) (Sigma Aldrich, M_{W} −31,000 Da −50,000 Da)
Thermoplastic polyurethane (PUR) (Merquinsa, Spain, Pearlbond ECO D 590)
Polyethylene glycol (PEG) (Sigma Aldrich, at different sizes: average Mₙ˜400, average Mₙ˜4,000 (“4K”) and average Mₙ˜20,000 (“20K”))

### Alpha Tocopherol Vitamin E (VI-E) (Sigma Aldrich, CAS no. 10191-41-0)

Polycarbophil (Konsyl pharmaceuticals, Inc.)

### Tributyl Acetyl Citrate (TAC) (Sigma Aldrich, CAS no. 77-90-7)

Biological glue: GLUBRAN® Synthetic Surgical Glue—(GEM, Itay)

### Tacker: AbsorbaTack™ Fixation Device—(Covidien)

### Hot Plate: 90 W Lead Free Digital Soldering Station Triple-7 T7-190DS-ESD

### Meshes: ProliteUltra (Atrium Medical)

Thermoplastic polyurethane (TEXIN DP7-3041 BMS, Bayer)

### Capa 6400 Polycaprolactone (Perstorp UK Limited)

### Example 1

### Preparation of Various Bioadhesive Compositions

### Polyvinylpyrrolidone and PCL:

Polyvinyl pyrrolidone (PVP) (at different Mw of: 10,000 Da, 40,000 Da and 60,000 Da), was dried at 50° C. under vacuum conditions overnight, and then mixed with various amounts of MMWPCL as detailed in Table 1 with or without tributyl acetyl citrate (TAC) and the mixture was melt blended at 80° C. to obtain the bioadhesive composition. Alternatively, solvent mixing using for example dichloromethane may be used to form the homogenous mixture.

### Fumaric Anhydride Oligomer (FAO) and PCL:

Initially, re-crystallization of fumaric acid (FA) was done by adding 5% FA (40 gram) to 95% ethanol (760 gram) and allowing the solution to evaporate in a vacuum chamber, at 50° C., for 10 hours. Complete dryness was obtained after 2 days at room temperature.

Acetic anhydride (500 ml) was heated to 150° C. in a sand pile on a hot plate magnetic stirrer. Then, 40 g of re-crystallized FA was added to the acetic anhydride and the system was allowed to stir for 2 hours, to obtain polymerization to fumaric anhydride oligomer (FAO).

After cooling, the system was allowed to crystallize under dark conditions at room temperature for about 2 weeks. The synthesized FAO was washed twice with toluene and evaporated under vacuum conditions for 3 days.

Five grams of the FAO were dissolved in 40 ml THF for an hour on a hot plate (˜50° C.-60° C.). Then, LMWPCL, HMWPCL or combinations of the two were added at various amounts as detailed in Table 1. Using this solvent based mixing procedure using magnetic stirring, a good, homogenous dispersion of FAO particles in the polymeric matrix of LMWPCL, HMWPCL or combinations, was achieved using THF as a solvent. The solvent was evaporated in a heated vacuum chamber for about 2-3 hours at 50° C. to total evaporation

FAO (between 2 to 4 grams) and MMWPCL (between 5.4 to 7.2 grams) were solvent blended using THF for 24 hours at room temperature and ultrasonic blended. The solvent was evaporated overnight and TAC was introduced and mixed using melt blending technique (85° C. for 1 hour)

### Polyvinyl Alcohol and PCL:

Polyvinyl alcohol (PVOH, Mw 89,000 Da −98,000 Da, 99+% hydrolyzed, from Sigma-Aldrich) is grinded into a fine particle powder using a laboratory grinder and is mixed with MMWPCL and TAC and melt blended (85° C. for 1 hour), in a ratio of 20% to 40% PVOH and 80% to 40% (90% PCL+10% TAC), until an homogeneous mixture is obtained.

### Thermoplastic Polyurethane and PCL:

Thermoplastic polyurethane (PUR) and MMWPCL (between 5.4 to 7.2 grams) were solvent blended using THF for 24 hours at room temperature and ultrasonic blended. The solvent was evaporated overnight and TAC was introduced and mixed using melt blending technique (85° C. for 1 hour).

In addition, VI-E, Polycarbophil-Konsyl, TAC or combinations were added to the bioadhesive compositions according to the amounts described in Table 1.

TABLE 1
Composition of the prepared bioadhesive samples

| Sample # | Composition (% wt) |
|---|---|
| 1 | 90% LMWPCL¹ + 10% FAO |
| 2 | 80% LMWPCL + 20% FAO |
| 3 | 70% LMWPCL + 30% FAO |
| 4 | 60% LMWPCL + 40% FAO |
| 5 | 75% LMWPCL + 20% FAO + 5% VI-E |
| 6 | 55% LMWPCL + 40% FAO + 5% VI-E |
| 7 | 95% LMWPCL + 5% VI-E |
| 8 | 80% LMWPCL + 20% FA |
| 9 | 75% LMWPCL + 20% FAO + 5% Polycarbophil |
| 10 | 100% LMWPCL |
| 11 | 95% LMWPCL + 5% TAC |
| 12 | 90% LMWPCL + 10% TAC |
| 13 | 75% LMWPCL + 20% FAO + 5% TAC |
| 14 | 70% LMWPCL + 20% FAO + 10% TAC |
| 15 | 75% LMWPCL + 25% HMWPCL² |
| 16 | 50% LMWPCL + 50% HMWPCL |
| 17 | 100% HMWPCL |
| 18 | 90% LMWPCL + 10% HMWPCL |
| 19 | Biological glue |
| 20 | Tacker |
| 21 | 72% MMWPCL³ + 20% FAO + 8% TAC |
| 22 | 63% MMWPCL + 30% FAO + 7% TAC |
| 23 | 54% MMWPCL + 40% FAO + 6% TAC |
| 24 | 72% MMWPCL + 20% PVOH + 8% TAC |
| 25 | 63% MMWPCL + 30% PVOH + 7% TAC |
| 26 | 54% MMWPCL + 40% PVOH + 6% TAC |
| 27 | 72% MMWPCL + 20% PUR + 8% TAC |
| 28 | 63% MMWPCL + 30% PUR + 7% TAC |
| 29 | 54% MMWPCL + 40% PUR + 6% TAC |
| 30 | 80% MMWPCL + 20% MMWPVP⁴ |
| 31 | 70% MMWPCL + 30% MMWPVP |
| 32 | 60% MMWPCL + 40% MMWPVP |
| 33 | 72% MMWPCL + 20% MMWPVP + 8% TAC |
| 34 | 63% MMWPCL + 30% MMWPVP + 7% TAC |
| 35 | 54% MMWPCL + 40% MMWPVP + 6% TAC |
| 36 | 100% MMWPCL |
| 37 | 25% LMWPCL + 75% HMWPCL |
| 38 | 95% LMWPCL + 5% PEG 400 |
| 39 | 90% LMWPCL + 10% PEG 400 |
| 40 | 75% LMWPCL + 25% PEG 400 |
| 41 | 95% LMWPCL + 5% PEG 4,000 |
| 42 | 90% LMWPCL + 10% PEG 4,000 |
| 43 | 75% LMWPCL + 25% PEG 4,000 |
| 44 | 95% (75% LMWPCL + 25% HMWPCL) + 5% PEG 4,000 |
| 45 | 97.5% (75% LMWPCL + 25% HMWPCL) + 2.5% PEG |
| | 4,000 |
| 46 | 95% (75% LMWPCL + 25% HMWPCL) + 5% TAC |
| 47 | 90% (75% LMWPCL + 25% HMWPCL) + 5% PEG 4,000 + |
| | 5% TAC |
| 48 | 95% (75% LMWPCL + 25% HMWPCL) + 5% PEG 20,000 |
| 49 | 50% MMWPCL + 50% LMWPCL |
| 50 | 95% MMWPCL + 5% PEG 4,000 |
| 51 | 90% MMWPCL + 5% PEG 4,000 + 5% TAC |
| 52 | 25% MMWPCL + 75% LMWPCL |
| 53 | 90% (50% MMWPCL + 50% LMWPCL) + |
| | 5% PEG 4,000 + 5% TAC |
| 54 | 100% Capa 6400 |
| 55 | 90% (25% MMWPCL + 75% LMWPCL) + |
| | 5% PEG 4,000 + 5% TAC |
| 56 | 80% (25% MMWPCL + 75% LMWPCL) + |
| | 10% PEG 4,000 + 10% TAC |
| 57 | 85% MMWPCL + 5% PEG 4,000 + 10% TAC |
| 58 | 85% MMWPCL + 10% PEG 4,000 + 5% TAC |
| 59 | 90% MMWPCL + 10% TAC |

¹LMWPCL = Low Molecular Weight PCL, Mn~10,000 Da;
²HMWPCL = High Molecular Weight PCL Mn~80,000 Da
³MMWPCL = Medium Molecular Weight PCL Mn~45,000 Da
⁴MMWPVP = Medium Molecular Weight PVP; M_{w}~40,000 Da

### Example 2

### DSC (Differential Scanning Calorimetry)

### Method:

Test conditions: heating and cooling of samples was performed at a rate of 20° C./minute. Differential scanning calorimetry or DSC is a thermoanalytical technique in which the difference in amount of heat required to increase the temperature of a sample and reference is measured as a function of the temperature. Using this technique it is possible to observe melting and crystallization events as well as the enthalpies of transitions.

### Results:

The results are summarized in Table 2.

TABLE 2
DSC results at temperature change rate of 20° C./min

| Sample | | | PCL ΔHₘ |
|---|---|---|---|
| # | Composition | Tₘ(° C.) | (J/g) |
| 2 | LMWPCL + 20% FAO | 61 | 95 |
| 5 | 75% LMWPCL + 20% FAO + 5% VI-E | 58 | 95 |
| 10 | LMWPCL | 68 | 101 |
| 11 | 95% LMWPCL + 5% TAC | 63 | 92 |
| 23 | 54% MMWPCL + 40% FAO + 6% TAC | 60 | 96 |
| 17 | HMWPCL | 67 | 74 |
| 26 | 54% MMWPCL + 40% PVOH + 6% TAC | 60 | 87 |
| 29 | 54% MMWPCL + 40% PUR + 6% TAC | 64 | 167 |
| 30 | 80% MMWPCL + 20% MMWPVP | 61 | 123 |
| 31 | 70% MMWPCL + 30% MMWPVP | 61 | 83 |
| 32 | 60% MMWPCL + 40% MMWPVP | 62 | 86 |
| 33 | 72% MMWPCL + 20% MMWPVP + | 61 | 117 |
| | 8% TAC | | |
| 34 | 63% MMWPCL + 30% MMWPVP + | 59 | 83 |
| | 7% TAC | | |
| 35 | 54% MMWPCL + 40% MMWPVP + | 59 | 74 |
| | 6% TAC | | |
| 36 | MMWPCL | 63 | 86 |

Comparison of different molecular weights of PCL, HMWPCL (Sample #18), LMWPCL (Sample #10) and MMWPCL (Sample #36) showed that HMWPCL has a lower degree of crystallization, compared to MMWPCL and LMWPCL, as reflected by the lower melt enthalpy of the HMWPCL, ΔHₘ (HMWPCL)=74 J/g vs. ΔHₘ (MMWPCL)=86 J/g and ΔHₘ (LMWPCL)=101 J/g. Cold crystallization was observed only for the LMWPCL.

Addition of FAO and TAC to LMWPCL reduce the crystallinity degree as reflected by the reduced melt enthalpy of ΔHₘ (LMWPCL)=101 J/g vs. ΔHₘ (75% PCL+20% FAO+5% VI-E)=95 J/g and increase a nucleating as evident by the increased T_{cc}; T_{cc} (LMWPCL)=14° C. vs. T_{cc} (75% LMWPCL+20% FAO+5% VI-E)=33° C.

Addition of fumaric anhydride oligomer (FAO) reduced the crystallinity degree of the low molecular weight PCL, reflected by the reduced melt enthalpy, from 101 J/g to 95 J/g.

Addition of tributyl acetyl citrate (TAC) as plasticizer also reduced the crystallinity degree of PCL from 101 J/g to 92 J/g.

Addition of either FAO or a combination of VI-E and TAC to PCL reduced the melting point; Tₘ (LMWPCL)=68° C. was decreased by adding FAO to Tₘ (LMWPCL+20% FAO)=61° C. or to Tₘ (75% LMWPCL+20% FAO+5% VI-E)=58° C.

Addition of polyvinyl pyrrolidone (PVP) reduced the crystallinity degree as reflected by the reduced melt enthalpy of ΔHₘ (MMWPCL)=86 J/g vs. ΔHₘ (70% MMWPCL+30% PVP)=83 J/g

The addition of TAC to the composition reduced the crystallinity degree even more, as reflected by the reduced melt enthalpy, ΔHₘ (60% MMWPCL+40% PVP)=86 J/g vs. ΔHₘ (50% MMWPCL+40% PVP+6% TAC)=74 J/g

The melting point of PCL was reduced with the addition of any one of FAO, TAC, PVOH and PVP.

For the purpose of applying the composition on the wound it is preferable that the melt enthalpy and melting point be low. In other words, a reduction in the degree of crystallinity, as reflected by a lower melting enthalpy as well as a lower melting point is highly desired, reducing the application temperature, energy and time.

Thus, in view of the above, the inventors have concluded that best results were observed for the formulation containing 40% PVP, 54% MMWPCL and 6% TAC with melting enthalpy of 74 J/g and a melting point of 59° C.

### Example 3

### Dynamic Mechanical Analyzer (DMA) Studies

### Method:

A sinusoidal stress is applied to a small rectangular sample using three point bending method and the strain in the material is measured, allowing one to determine the storage and loss modulus. The temperature of the sample was varied from 20 to 550 C, leading to variations in the storage and loss modulus; this approach was used to locate the glass transition temperature of the material, as well as to identify transitions corresponding to other molecular motions. The samples were analyzed using frequency of 1 Hz.

### Results:

Table 3 summarizes the DMA results obtained for the different compositions. The storage modulus at two relevant temperatures: 23° C. (room temperature) and 37° C. (human body temperature) is summarized in Table 3 for the different compositions.

TABLE 3
DMA results

| | | storage | storage |
|---|---|---|---|
| | | modulus | modulus |
| Sample# | Composition | at 23° C. | at 37° C. |
| 1 | 90% LMWPCL + 10% FAO | 436 | 387 |
| 2 | 80% LMWPCL + 20% FAO | 584 | 508 |
| 3 | 70% LMWPCL + 30% FAO | 955 | 838 |
| 4 | 60% LMWPCL + 40% FAO | 1230 | 1090 |
| 5 | 75% LMWPCL + 20% FAO + | 802 | 702 |
| | 5% VI-E | | |
| 6 | 55% LMWPCL + 40% FAO + | 1361 | 1214 |
| | 5% VI-E | | |
| 7 | 95% LMWPCL + 5% VI-E | 392 | 340 |
| 8 | 80% LMWPCL + 20% FA | 825 | 699 |
| 9 | 75% LMWPCL + 20% FAO + | 706 | 622 |
| | 5% KONSYL | | |
| 10 | 100% LMWPCL | 434 | 338 |
| 11 | 95% LMWPCL + 5% TAC | 272 | 231 |
| 12 | 90% LMWPCL + 10% TAC | 263 | 221 |
| 15 | 75% LMWPCL + 25% HMWPCL | 327 | 276 |
| 16 | 50% LMWPCL + 50% HMWPCL | 382 | 317 |
| 17 | 100% HMWPCL | 315 | 261 |
| 18 | 90% LMWPCL + 10% HMWPCL | 428 | 364 |
| 30 | 80% MMWPCL + 20% PVP | 489 | 377 |
| 31 | 70% MMWPCL + 30% PVP | 171 | 129 |
| 32 | 60% MMWPCL + 40% PVP | 540 | 434 |
| 33 | 72% MMWPCL + 20% PVP + | 230 | 166 |
| | 8% TAC | | |
| 34 | 63% MMWPCL + 30% PVP + | 155 | 117 |
| | 7% TAC | | |
| 35 | 54% MMWPCL + 40% PVP + | 87 | 67 |
| | 6% TAC | | |
| 36 | 100% MMWPCL | 405 | 302 |

The storage modulus at two temperature, 23° C. and 37° C. is obtained as a function of the adhesive composition comparison of the average storage modulus at 37° C. between LMWPCL (sample #10) and HMWPCL (sample #17) show that LMWPCL had an average storage modulus at 37° C. of 338 which is higher compared to the average storage modulus at 37° C. of HMWPCL of 261, thereby leading to high brittleness of the LMWPCL and a higher stiffness.

The addition of FAO to LMWPCL (samples 1-4) gradually increased the average storage modulus at 23° C. from 434 to 436 (10% FAO), 584 (20% FAO), 955 (30% FAO), 1230 (40% FAO) and also at 37° C. from 338 to 387 (10% FAO), 508 (20% FAO), 838 (30% FAO), 1090 (40% FAO), thereby increasing the brittleness of LMWPCL and the stiffness.

These results indicate that addition of additives to the composition increased the storage modulus.

The addition of VI-E to LMWPCL+FAO (sample 5 vs. sample 2 and sample 6 vs. sample 4) also increased the brittleness as indicated by the increasing storage modulus at 23° C. and 37° C.

Also, addition of Konsyl to LMWPCL+FAO (sample 9 vs. sample 2) increased the brittleness as indicated by storage modulus at 23° C. and 37° C.

It can be seen that the addition of plasticizer, TAC, reduced the stiffness and brittleness

However, when VI-E is added to LMWPCL without FAO (sample 7 vs. sample 5), VI-E works as plasticizer as indicated by the lower storage modulus at 23° C. and 37° C.

Also, when TAC was added to LMWPCL it had a plasticizing effect as indicated by the storage modulus being lower in the presence of TAC (samples 11 and 12 vs. sample 10). No significant change in storage modulus was observed when TAC was added at 5% or 10% TAC, thus it was concluded that adding 5% TAC is sufficient to obtain a plasticizing effect.

Mixing HMWPCL with LMWPCL lead to a significant reduction of the storage modulus, with the best results obtained at a combination of 75% LMWPCL and 25% HMWPCL (sample 15).

Based on these studies, it was concluded that storage modulus can be reduced by mixing 25% HMWPCL with 75% LMWPCL. In addition, 5% TAC or 10% TAC was useful for reducing the storage modulus of the composition.

The addition of PVP to the PCL did not increase dramatically the storage modulus and the reduction of the stiffness by TAC in compositions with PVP was more meaningful, leading to very soft formulations with very low storage modulus, as the amount of PVP increased.

The above results suggest that for the purpose of the present invention a desired storage modulus at room temperature and/or body temperature is within the range of 100 to 600, with preferably no significant change between the two temperatures. Thus, when considering the combination of the thermoplastic polymeric matrix forming the composition of the invention it was concluded that while the polymer may be combined with additives, such as plasticizers, the amount of the plasticizer should not be more than 10% of the thermoplastic portion of the composition.

### Example 4

### In Vitro Adhesion Tests

For determining the strength of adhesion of the bioadhesive composition to biological tissue, a standard test method for strength properties of tissue adhesives in T-Peel by tension loading was used (ASTM F2256).

### Testing:

Characterizing the properties of the tested adhesive device in combination with biological tissue was conducted under conditions similar to that of the human body, i.e. preferably in a bath, but alternatively in a chamber that is set to 37° C. The biological tissue was chicken breast.

Various bioadhesive samples as detailed in Table 4 below were applied as a melt coating onto stripes of commercial hernia mesh (made of polypropylene) as detailed below:

1. Polypropylene Monofilament meshes were cut into strips of 20×50 mm.

2. Films from each composition (Table 4) were prepared using hot press (80° C., 5 minutes). Square patches of 20×20×0.4 mm were cut from each composition.

3. The composition patches were hot melted an adhered to the mesh strips, using a 20×20 mm flat tip hot plate covered with Teflon fabric, heated to 90° C., for 20 seconds.

4. Chicken breast tissue were cut into 30×30 mm pieces and were fixed into a U apparatus (as described below) using steel wires.

5. The meshes were hot melted and glued to the pieces of chicken breast tissue samples using hot plate tip, heated to 90° C., for 30 seconds.

6. Peel strength was determined by peeling the mesh from the chicken breast tissue samples, using an Instron testing machine (INSTRON 4481), specially adapted to hold the tissue, while the upper grip held one side of the adhered device. Low capacity load cell (100 N) was used since adhesive strength forces tend to be under 10 or 20 N. Peel Test conditions of INSTRON 4481 (Load cell—100N): Crosshead speed—5 mm/min.

A dedicated fixation base was designed and developed in order to match the tensile test machine (‘Instron’) specifications. A small wood fixture in a U form was coated with aluminum adhesive tape. Small holes were made in the wood fixture in order to fix the small chicken breast samples using steel wire. After fixing the chicken breast samples on the wood fixture the mixture itself was fixed on the basis of the test machine using a small magnet.

The bioadhesive devices comprising the various combinations described above were adhered onto a tissue. A hot plate device was set to a temperature of 80° C. The adhesion strength of the various devices was tested after 10 minutes, at room temperature.

In addition, the properties of the tested adhesive device were tested in combination with thermoplastic polyurethane used as a tissue equivalent stimulant. These experiments were conducted as follows:

1. Plates from thermoplastic polyurethane based on TEXIN DP7-3041 BMS, Bayer were prepared using hot press, strips (20×80×2 mm) were cut.

2. Polypropylene meshes were cut into strips of 20×80 mm.

3. Films from each composition (Table 5) were prepared using hot press (80° C., 5 minutes). Square patches of 20×20×0.4 mm were cut from each composition film.

4. The composition patches were hot melted an adhered onto the polyurethanes strips in a sandwich structure where the composition patches were occluded between the mesh strip and the polyurethane strip, using a 20×20 mm flat tip hot plate covered with Teflon fabric, heated to 90° C., for 30 seconds.

5. Peel strength was determined by peeling the mesh from the tissue equivalent stimulant, using an Instron 4481, using the following condition tests: load cell: 100N, 50 mm/min strain rate.

### Results:

The adhesion strength of the different samples to chicken breast are summarized in Table 4.

TABLE 4
adhesion strength to chicken breast

| | | Load at | | | |
|---|---|---|---|---|---|
| | | max. load | Load/Width at | adhesion area | adhesion strength |
| Sample# | Composition | (N) | max. load (N/mm) | (cm²) | (gr/cm²) |
| 1 | 90% LMWPCL + 10% FAO | 0.69 | 0.045 | 7.3 | 23 |
| 2 | 80% LMWPCL + 20% FAO | 0.69 | 0.045 | 7.3 | 23 |
| 3 | 70% LMWPCL + 30% FAO | 0.62 | 0.041 | 7.0 | 20.7 |
| 4 | 60% LMWPCL + 40% FAO | 0.66 | 0.046 | 6.2 | 22 |
| 5 | 75% LMWPCL + 20% FAO + 5% VI- | 0.67 | 0.040 | 8.3 | 22.3 |
| | E | | | | |
| 8 | 80% LMWPCL + 20% FA | 0.37 | 0.023 | 7.6 | 12.3 |
| 9 | 75% LMWPCL + 20% FAO + 5% | 0.72 | 0.052 | 6.0 | 24.0 |
| | Polycarbophil | | | | |
| 10 | LMWPCL | 0.06 | 0.004 | 7.6 | 2 |
| 19 | Biological glue | 0.89 | 0.060 | 6.7 | 29.7 |
| 20 | Tacker-(1 unit) | 0.24 | NA | NA | NA |
| 30 | 80% MMWPCL + 20% MMWPVP | 3.5 | 0.175 | | 87.5 |
| 31 | 70% MMWPCL + 30% MMWPVP | 1.45 | 0.073 | | 36.3 |
| 32 | 60% MMWPCL + 40% MMWPVP | 0.93 | 0.047 | | 23.3 |
| 33 | 72% MMWPCL + 20% MMWPVP + | 0.72 | 0.036 | | 18.0 |
| | 8% TAC | | | | |
| 34 | 63% MMWPCL + 30% MMWPVP + | 0.96 | 0.048 | | 24.0 |
| | 7% TAC | | | | |
| 35 | 54% MMWPCL + 40% MMWPVP + | 0.48 | 0.024 | | 12.0 |
| | 6% TAC | | | | |

As indicated from Table 4, shows adhesion strength values of the adhesive devices to chicken breast, addition of FAO (samples 1-4), specifically low concentrations of FAO (samples 1 and 2, up to 20%) improved the adhesion to the biological tissue. The addition of Vitamin E (sample #5) did not increase the adhesion strength. The addition of Polycarbophil (Konsyl) to a composition of PCL and FAO (sample #9) increased the adhesion strength in chicken breasts.

It could be seen that PCL alone cannot lead to sufficient adhesion to the tissue (formulation #10) and it requires the addition of bioadhesive, such as FAO or PVP.

Addition of PVP to the compositions without plasticizer (such as TAC) has shown good adhesion strength, for example sample #30 (20% MMWPVP) and sample #31 (30% MMWPVP). While not included in Table 4, it is noted that the inventors have found that LMWPVP, when combined with MMWPCL (at a ratio of 20 to 40%) did not show any adhesion properties. Thus, it was concluded that medium weight PVP is preferred among the various PVP tested.

Further, the compositions described in Table 4 were compared to a commercial tacker and to a liquid commercial glue. It can be seen that the tacker was easily removed from the tissue (showed no adhesion) while the compositions exemplified were as good (if not better) than the biological glue.

The adhesion strength of the different compositions to the thermoplastic polyurethane used as a tissue equivalent stimulant are summarized in Table 5 (Peel tests)

TABLE 5
adhesion strength to thermoplastic polyurethane

| | | | | Load/width | |
|---|---|---|---|---|---|
| Sample | | Adhesion Failure | Load at | at max | Avg load |
| # | Composition | description | max (N) | load (N/mm) | (N/mm) |
| 21 | 72% MMWPCL + 20% FAO + 8% TAC | cohesive | 13.6 | 0.68 | 0.47 |
| 22 | 63% MMWPCL + 30% FAO + 7% TAC | cohesive | 31.2 | 1.56 | 1.23 |
| 23 | 54% MMWPCL + 40% FAO + 6% TAC | cohesive | 29.0 | 1.45 | 1.11 |
| 24 | 72% MMWPCL + 20% PVOH + 8% TAC | cohesive | 46.2 | 2.31 | 1.73 |
| 25 | 63% MMWPCL + 30% PVOH + 7% TAC | cohesive | 37.8 | 1.89 | 1.19 |
| 26 | 54% MMWPCL + 40% PVOH + 6% TAC | cohesive | 48.6 | 2.43 | 1.84 |
| 27 | 72% MMWPCL + 20% PUR + 8% TAC | adhesive/cohesive | 45.8 | 2.29 | 1.61 |
| 28 | 63% MMWPCL + 30% PUR + 7% TAC | adhesive/cohesive | 54.4 | 2.72 | 1.67 |
| 29 | 54% MMWPCL + 40% PUR + 6% TAC | adhesive/cohesive | 45.5 | 2.28 | 1.21 |
| 30 | 80% MMWPCL + 20% MMWPVP | cohesive | 34.5 | 1.72 | 1.19 |
| 31 | 70% MMWPCL + 30% MMWPVP | cohesive | 25.1 | 1.25 | 0.87 |
| 32 | 60% MMWPCL + 40% MMWPVP | adhesive/cohesive | 12.7 | 0.64 | 0.40 |
| 33 | 72% MMWPCL + 20% MMWPVP + 8% TAC | adhesive/cohesive | 26.0 | 1.30 | 1.01 |
| 34 | 63% MMWPCL + 30% MMWPVP + 7% TAC | adhesive/cohesive | 17.7 | 0.88 | 0.61 |
| 35 | 54% MMWPCL + 40% MMWPVP + 6% TAC | cohesive | 31.4 | 1.57 | 1.15 |

Adhesion Failure Description depicts a qualitative analysis of the adhesive in terms of Cohesive or Adhesive failure (fracture). The failure of an adhesive can be classified into two types: Adhesive Failure (interfactial fracture), where failure occurs at the interface between the adhesive and the substrate (such as in polyurethane tape) or Cohesive Failure, where the failure occurs within the center of the adhesive material, indicating a good adhesion to the substrate. Mixed Cohesive/Adhesive failure can also occur, indicating a partial adhesion to the substrate.

As could be seen from Table 5, good adhesion results were obtained when using FAO, PUR, PVOH and PVP compositions, specially 30% FAO with TAC (1.56 N/mm), 40% PVOH with TAC (2.43 N/mm), all combinations with PUR, 20% PVP (34.5 N/mm) and PVP with TAC (1.57 N/mm). However, it is noted that the good results with PUR and probably with PVOH are due to the specific synthetic substrate used (polyurethane plate). Thus, for further investigation, PVP or FAO were used.

### Example 5

### Rheology Test

### Method:

Rheology test using DMA Parallel Plate Dynamic Rheology was performed in order to evaluate the melt viscosity of the different thermoplastic polymers (without the bioadhesive polymer). In this connection, it is noted that the thermoplastic polymers acts as a polymeric matrix for the bioadhesive polymer and it is preferable that the matrix fluidizes so as to allow “release” of the bioadhesive polymer for adherence to the biological tissue.

Accordingly, a parallel plate rheometer (AR-G2 TA Instruments) was used for the complex viscosity determination using torsion mode, frequency sweep step (1 to 10 Hz), samples of 25 mm diameter, 2 mm thickness and test temperature: 85° C. The complex viscosity was obtained as a function of the frequency and the initial point was recorded and is shown in the Table 6.

### Results:

An important property for thermally responsive adhesives is the melt flow rheology. In order to optimize the melt flow and determine the composition with the lowest (preferred) melt viscosity and yet with high tensile strength and toughness three different molecular weights of PCL (10K, 45K and 80K) were used with different additives (TAC and PEG 400, PEG 4K and PEG 20K), typically referred to as plasticizer.

The tensile tests results were obtained from DMA-Q800 TA Instruments, using the static tensile test mode. The results of tensile strain and strength can be seen in Table 6.

TABLE 6
Parallel Plate rheometer and tensile tests results

| | | | Tensile | Tensile |
|---|---|---|---|---|
| Sample | | complex | strain | strength |
| # | Composition | viscosity | (%) | (MPa) |
| 37 | 25% LMWPCL + 75% HMWPCL | 500 | 3.1 | 10.6 |
| 16 | 50% LMWPCL + 50% HMWPCL | 182 | 2.4 | 8.9 |
| 38 | 95% LMWPCL + 5% PEG 400 | 8 | 0.8 | 2.2 |
| 39 | 90% LMWPCL + 10% PEG 400 | 8 | 0.75 | 2.3 |
| 40 | 75% LMWPCL + 25% PEG 400 | 5 | 1 | 1.8 |
| 41 | 95% LMWPCL + 5% PEG 4K | 10 | 1.2 | 3.7 |
| 42 | 90% LMWPCL + 10% PEG 4K | 9 | 1 | 3.9 |
| 43 | 75% LMWPCL + 25% PEG 4K | 11 | 1 | 4.8 |
| 10 | 100% LMWPCL | 68 | 2 | 8.8 |
| 15 | 75% LMWPCL + 25% HMWPCL | 61 | 3.3 | 8.6 |
| 44 | 95% (75% LMWPC + 25% | 247 | 1.1 | 4.6 |
| | HMWPCL) + 5% PEG 4K | | | |
| 45 | 97.5% (75% LMWPCL + 25% | 64 | 0.9 | 3.3 |
| | HMWPCL) + 2.5% PEG 4K | | | |
| 46 | 95% (75% LMWPCL + 25% | 52 | 1.9 | 4.8 |
| | HMWPCL) + 5% TAC | | | |
| 47 | 90%(75% LMWPCL + 25% | 163 | 4 | 12 |
| | HMWPCL) + 5% PEG4K + | | | |
| | 5% TAC | | | |
| 48 | 95%(75% LMWPCL + 25% | 91 | 2.5 | 10.8 |
| | HMWPCL) + 5% PEG 20K | | | |
| 36 | 100% MMWPCL | 221 | 2.5 | 9.3 |
| 49 | 50% MMWPCL + 50% LMWPCL | | 3.3 | 9.2 |
| 50 | 95% MMWPCL + 5% PEG 4K | 204 | | |
| 51 | 90% MMWPCL + 5% PEG 4K + | 172 | 5.5 | 11 |
| | 5% TAC | | | |
| 52 | 25% MMWPCL + 75% LMWPCL | 34 | 3.8 | 7 |
| 53 | 90%(50% MMWPCL + 50% | 41 | 2.1 | 6.4 |
| | LMWPCL) + 5% PEG 4K + | | | |
| | 5% TAC | | | |
| 54 | 100% Capa 6400 | 531 | 3.1 | 7.2 |
| 55 | 90%(25% MMWPCL + 75% | 17 | 1.7 | 5 |
| | LMWPCL) + 5% PEG4K + | | | |
| | 5% TAC | | | |
| 56 | 80%(25% MMWPCL + 75% | 16 | 1.5 | 2.7 |
| | LMWPCL)+ 10% PEG4K + | | | |
| | 10% TAC | | | |
| 57 | 85% MMWPCL + 5% PEG4 + | 105 | 5.6 | 8.4 |
| | 10% TAC | | | |
| 58 | 85% MMWPCL + 10% PEG4K + | 200 | 4.3 | 9.2 |
| | 5% TAC | | | |
| 59 | 90% MMWPCL + 10% TAC | 90 | 5.6 | 8.1 |

While not shown in Table 6, it was further observed by the inventors that a mixture of HMWPCL and LMWPCL had a very high complex viscosity (500) and LMWPCL (Formulation #10) had a low complex viscosity (68). The less preferable viscosities were improved by the addition of a small amount of PEG (400, 4K or 20K).

The lowest value of complex viscosity (5) was obtained for 75% LMWPCL+25% PEG 400, however this formulation was considered brittle, according to the tensile strain of 1%, thus unfavorable.

Compositions of high and low molecular weight PCL have shown relatively versatile values of complex viscosity depending on the ratio between the low and high MW PCL. When the LMWPCL was high, the values of complex viscosity were low, but the resulting matrices were brittle due to LMWPCL influence.

Optimal results were obtained using MMWPCL or a mixture of LMWPCL and HMWPCL with an average MW of medium size (i.e. average Mw of 40,000 to 100,000).

A good balance of relatively low viscosity, toughness and tensile strength could be found with the composition based on 90% MMWPCL and 10% TAC (Complex viscosity of 90, tensile strain of 5.6% and tensile strength of 8.1 MPa). This led the inventors to the conclusion that for the thermoplastic polymer forming the polymeric matrix of the composition would preferably include a medium molecular weight polymer or a combination of polymers providing an average molecular weight in the average weight, optionally combined with no more than 10% additive.

### Example 6

### Determination of FAO Molecular-Weight

Molecular weight of synthesized FAO can be analyzed using two methodologies:

(1) End group analysis using titration for determining Mn; and

(2) End-group analysis by H1-NMR.

### Example 7

### In Vivo Test

Male Sprague-Dawley rats, weighing about 400-450 g, will be anesthetized using ketamine and xylazine (85 mg/kg ketamine, 5 mg/kg xylazine). After the abdomen will be shaved, a longitudinal skin incision will be made along the linea alba and subcutaneous fat tissue. Two pieces of about 1.5 cm in diameter of the fascia will be dissected with a scalpel and will be detached from the underlying muscle. This will be performed in the right and left abdominal rectus muscles, 1.5 cm below the rib cage and 1.5 cm laterally to the linea alba.

Meshes will be cut prior to implantation, under sterile conditions, to a size of 2×2 cm. Lesions will be covered with 2×2 cm sterile mesh coated with one composition and afterwards the meshes will be secured to the facial or to the muscle by thermal fixation procedures using Hotplate.

The skin incision will be closed with non-resorbable suture material. Rats will be checked daily for signs of inflammation, delayed wound healing, pain or herniation.

Rats will be sacrificed in deep anesthesia on the 1th day and on the 7th post operatively by CO₂. (1 per each group respectively) The skin incision will be re-opened and the meshes will be removed together with the musculature and will be taken for mechanical test.

1.-53. (canceled)

54. A bioadhesive composition, comprising: a polymeric matrix; and at least one synthetic bioadhesive polymer carried by the polymeric matrix, the polymeric matrix comprising at least one synthetic thermoplastic polymer characterized by one or more of the following
an average molecular weight in the range of from 20,000 Da to 90,000 Da, and
it comprises polycaprolactone (PCL),
wherein exposure to heat causes the bioadhesive composition to transform into a non-solid state and to cohesively adhere to a biological tissue upon subsequent cooling thereof.

55. The bioadhesive composition of claim 54, wherein the at least one thermoplastic polymer consists of a single type of polymer having an average molecular weight in the range of from 40,000 to 60,000 or a combination of two or more thermoplastic polymers having an average molecular weight in the range of from 40,000 to 60,000.

56. The bioadhesive composition of claim 55, wherein the at least one thermoplastic polymer comprises or consists of PCL.

57. The bioadhesive composition of claim 56, wherein the PCL has an average molecular weight in the range of from 43,000 to 48,000.

58. The bioadhesive composition of claim 54, wherein the thermoplastic matrix has a complex viscosity in the range of from 50 to 600 determined at a frequency sweep step of 1 to 10 Hz and at a test temperature of 85° C.

59. The bioadhesive composition of claim 54, wherein the synthetic bioadhesive polymer comprises PVP.

60. The bioadhesive composition of claim 54, which transforms into a non-solid state at a temperature between 50° C. and 100° C.

61. A method for producing a bioadhesive composition, comprising: mixing a mixture comprising at least one synthetic thermoplastic polymer suitable for forming a polymeric matrix and at least one synthetic bioadhesive polymer under conditions at which the at least one synthetic thermoplastic polymer transforms into a non-solid state, the at least one synthetic thermoplastic polymer being characterized by one or more of the following
an average molecular weight in the range of from 20,000 Da to 90,000 Da;
it comprises polycaprolactone; and
the bioadhesive composition being such that when in a non-solid state it is capable of cohesively adhering to a biological tissue.

62. The method of claim 61, wherein the at least one thermoplastic polymer consists of a single type of polymer having an average molecular weight in the range of from 40,000 to 60,000 or a combination of two or more thermoplastic polymers having an average molecular weight in the range of from 40,000 to 60,000.

63. The method of claim 62, wherein the at least one thermoplastic polymer comprises or consists of PCL.

64. The method of claim 63, wherein the PCL has an average molecular weight in the range of from 43,000 to 48,000.

65. The method of claim 61, wherein the polymeric matrix constitutes of from 60% to 80% (w/w) of the composition.

66. The method of claim 61, wherein the thermoplastic matrix has a complex viscosity in the range of from 50 to 600 determined at a frequency sweep step of 1 to 10 Hz and at a test temperature of 85° C.

67. The method of claim 61, wherein the synthetic bioadhesive polymer comprises PVP.

68. The method of claim 61, wherein the conditions comprise mixing of a mixture at a temperature of from 50° C. to 100° C. to form an essentially homogenous flowing mixture and cooling the essentially homogenous flowing mixture to form a solid bioadhesive composition.

69. The method of claim 68, wherein said cooling is on a support structure, in a mold or in an applicator.

70. A bioadhesive device comprising a support structure and a bioadhesive composition according to claim 54, wherein at least a portion of the support structure is coated or impregnated with the bioadhesive composition.

71. A therapeutic method, comprising: placing a bioadhesive composition as claimed in claim 54, optionally on a support structure, in a mold or in an applicator, onto or within a biological tissue region, the tissue region comprising tissue damage or a region in predisposition of developing tissue damage; wherein placing of the bioadhesive composition or the bioadhesive device comprises allowing contact of the bioadhesive composition with the biological tissue region while the bioadhesive composition is heated to a temperature that causes the composition to be in a non-solid state and once in contact with the biological tissue region, allowing the bioadhesive composition to cool to the temperature of the biological tissue region thereby providing cohesive adherence of the bioadhesive composition or the bioadhesive device to the biological tissue region.

72. The method of claim 71, wherein the bioadhesive composition is heated immediately before or during application thereof onto the biological tissue region, thereby placing the bioadhesive composition onto the biological tissue region while in non-solid state.

73. The method of claim 72, wherein heating comprises applying dielectric heat.
